(19) United States
(12) Patent Application Publication
(10) Pub. No.: US 2022/0401677 A1
(43) Pub. Date: Dec. 22, 2022

(54) ANTIMICROBIAL LIGHT-EMITTING OROPHARYNGEAL DEVICE FOR VENTILATOR-ASSOCIATED PNEUMONIA

(71) Applicant: Lumen Catheters, LLC, Marlborough, MA (US)

(72) Inventors: Adam E.M. Eltorai, Marlborough, MA (US); Walter Shunaula, Shelton, CT (US); Paul DiCesare, Shelton, CT (US)

(21) Appl. No.: 17/851,562

(22) Filed: Jun. 28, 2022

Related U.S. Application Data
(63) Continuation-in-part of application No. 17/665,965, filed on Feb. 7, 2022.
(60) Provisional application No. 63/146,697, filed on Feb. 7, 2021.
(60) Provisional application No. 63/215,910, filed on Jun. 28, 2021.

(51) Int. Cl. A61M 16/04

(52) U.S. Cl. CPC A61M 16/0402; A61M 2209/10; A61N 2005/0652

(57) ABSTRACT

A method of reducing the risk of ventilator-associated pneumonia by decontaminating the oropharynx using a safe, antimicrobial light-emitting device.

### RELATED APPLICATIONS

This Patent Application claims the benefit under 35 U.S.C. § 119(e) of U.S. Provisional Patent App. No. 63,215,910, filed Jun. 28, 2021, entitled “ANTIMICROBIAL LIGHT-EMITTING DEVICES,” and is a Continuation-in-Part (CIP) of U.S. patent application Ser. No. 17/665,965, filed Feb. 7, 2022, entitled “ANTIMICROBIAL LIGHT-EMITTING PERCUTANEOUS SITE DRESSING,” which claims the benefit under 35 U.S.C. § 119(e) of U.S. Provisional Patent App. No. 63,146,697, filed Feb. 7, 2021, entitled “ANTIMICROBIAL LIGHT DRESSING DEVICE,” incorporated herein by reference in entirety.

### BACKGROUND

Healthcare providers often access treatment areas through the use of elongated devices that penetrate or pierce a physiological boundary, such as the skin/epidermal system, gastrointestinal, urinary, nasal and ocular, to name several. The penetration of a foreign member introduces a risk of adverse results from infection resulting from the artificial path created by the inserted foreign member. Particularly in a healthcare environment, where many therapeutic procedures utilize these foreign members, the risk of provider or hospital caused infections is prevalent.

The primary route of infection of the lungs is through microaspiration of organisms that have colonized the oropharyngeal tract (or, to a lesser extent, the gastrointestinal tract). Approximately 45 percent of healthy subjects aspirate during sleep and an even higher proportion of severely ill patients aspirate routinely. Although frequently regarded as partially protective, the presence of an endotracheal tube facilitates aspiration of oropharyngeal secretions and bacteria into the lungs. Depending upon the number and virulence of organisms reaching the lung and the host response, pneumonia may ensue.

Hospitalized patients often become colonized with microorganisms acquired from the hospital environment, and as many as 75 percent of severely ill patients will be colonized within 48 hours. An additional mechanism of inoculation in mechanically ventilated patients is direct contact with environmental reservoirs, including respiratory devices and contaminated water reservoirs. Disposable tubing used in respiratory circuits or tracheostomy or endotracheal tubes may become contaminated in the process of routine nursing care or via the (contaminated) hands of hospital personnel. Such contamination can occur despite rigorous cleaning of ventilator equipment. Resultant pneumonia is called ventilator-associate pneumonia (VAP).

Decontamination of the oropharynx and digestive tract has been shown to reduce VAP.

### SUMMARY

Configurations herein are based, in part, on the observation that therapeutic procedures and treatment often involve a foreign member for transfer of fluids or samples between the human patient body and a treatment source or testing facility.

Unfortunately, conventional approaches often involve the use of a foreign member such as a needle, vessel or probe to cross an external bodily boundary to access various organ systems for used in patient care. Insertion or breach into the bodily region by these foreign members can form a path for pathogens such as bacteria and other microorganisms to cause infection. Accordingly, configurations herein substantially overcome the shortcomings of the infection risk presented by conventional foreign members by providing an antibacterial, antipathogen light source for illuminating or irradiating a treatment region defining an insertion point of epidermal, gastrointestinal, urinary, or oral breach by a foreign member used in the course of treatment.

Oropharyngeal devices are rigid or semi-rigid elongated tubular structures adapted for insertion onto the human throat and airway for establishing a fluidic path for respiration, often in an exigent or emergency situation for establishing an airway. Typical scenarios include bag-mask ventilation, extreme sleep apnea, or other remediation of a reduced or constricted airway. In general, oropharyngeal devices have a stiff curvature to conform to the tongue and displace it away from the posterior pharyngeal wall, thereby restoring pharyngeal airway volume. Particularly in the case of ventilation usage, the oropharyngeal device may remain for an extended period, creating a path of vulnerability for pathogens and infection.

Other configuration address devices for percutaneous delivery. In the case of percutaneous breach, intravenous delivery of medication is an effective medium for medicinal treatment directly to blood or tissue, which allows the medication to be quickly delivered to a specific region. General bloodstream delivery avoids degradation that can occur by oral administration which must pass via the gastrointestinal barrier. Unfortunately, conventional approaches to percutaneous delivery, typically via a needle or similar insertion member, suffer from the shortcoming that they pose an infection risk from a breach of the natural dermal (skin) barrier which guards against infiltration of pathogens. Typically, an antimicrobial substance is applied around the insertion point of the needle, however such chemical based approaches generally have diminishing effects over time, and need repeated applications for continued effectiveness.

Accordingly, configurations herein substantially overcome the shortcomings of chemical and topical approaches by providing an antimicrobial light device, system and method for a percutaneous treatment that bathes a treatment region around the percutaneous insertion with an antibacterial illumination source for preventing pathogens around the insertion from entering via the dermal puncture created by the insertion. The antimicrobial light dressing device combines a circumferential body centered around the insertion, and an arrangement of LEDs around the body that focus the light around the insertion and onto a therapeutic region of the insertion. An opening in the circumferential body has an articulated protrusion for offsetting a medicinal vessel such as an IV tube off the skin surface to avoid blocking light to an area under the vessel. The result is a 360 degree coverage of antimicrobial light around the percutaneous insertion as the medicinal vessel contacts the skin surface only at the insertion point in the center of the treatment region.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context view of a medical treatment environment suitable for use with configurations herein;

FIG. 2 is a perspective view of the medical device for antimicrobial light treatment of a percutaneous insertion site;

FIGS. 3A-3C show engagement of the device of FIG. 2 with a treatment region defined by the percutaneous insertion site;

FIGS. 4A-4B show perspective views of a central void in the device of FIGS. 1-3c;

FIG. 5 shows a plan view of the device of FIGS. 1-4B;

FIG. 6 shows a side, cutaway view of the central void and illumination cavity formed by the device of FIGS. 1-5;

FIG. 7 shows a perspective view of the illumination cavity of FIG. 6;

FIG. 8 shows a bottom view of the device of FIGS. 1-7;

FIG. 9 shows an underside perspective view of the device and illumination/light cavity of FIGS. 6-8;

FIG. 10 shows a method of applying the antimicrobial light treatment of FIGS. 1-9;

FIG. 11 shows an alternative configuration of the safety device for use with an oropharyngeal device;

FIG. 12 is a perspective view of the oropharyngeal device of FIG. 11;

FIG. 13 is a side view of a deployed device as in FIGS. 11-12;

FIG. 14 is a side transparent view of the illumination sources of FIGS. 11-13; and

FIG. 15 is a side schematic view showing axial orientation of the device as in FIGS. 11-14.

### DETAILED DESCRIPTION

A device for the dressing of wounds and insertion sites of percutaneous and drug delivery devices provides circumferential protection of a wound or insertion site of a percutaneous or drug delivery device. In particular, the device is an integrated dressing for vascular and non-vascular percutaneous medical devices (e.g., IV catheters, central venous lines, arterial catheters, dialysis catheters, peripherally inserted coronary catheters, mid-line catheter, drains, chest tubes, externally placed orthopedic pins, ventricular assist device drivelines, and epidural catheters) comprising an adhesive dressing and an antimicrobial light source, such as visible light, far UVC light, and any suitable electromagnetic emission of a therapeutically beneficial wavelength. The dressing device reduces infection risk from vascular and non-vascular percutaneous medical devices by providing sufficient tissue-safe antimicrobial light at a wound or insertion site.

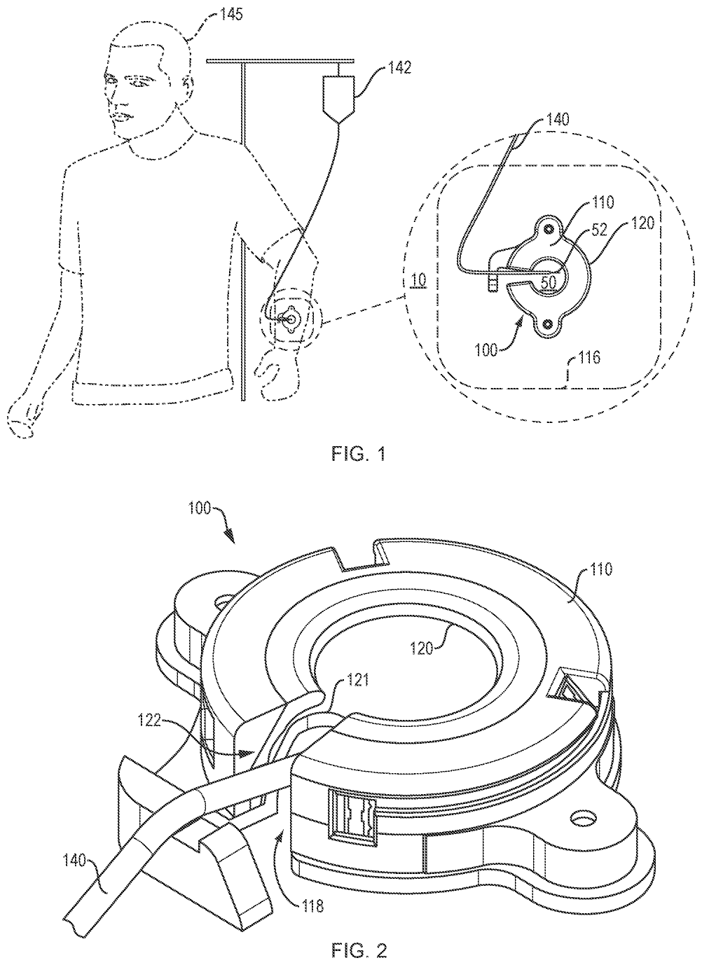

FIG. 1 is a context view of a medical treatment environment suitable for use with configurations herein. Referring to FIG. 1, an antimicrobial epidermal device 100 includes a circumferential light-emitting body 110 configured for adhesion around a percutaneous insertion site 52 for directing therapeutic light at the percutaneous insertion site while permitting unobstructed passage of a medication vessel 140 to the percutaneous insertion site 52. The medication vessel emanates from a fluidic source 142 of medication or other liquid, such as an IV (Intravenous) bag. The percutaneous insertion site 52 defines a surrounding treatment region 50, typically on an arm of a patient 145 because of ease of IV access, however any suitable epidermal region may be selected for the percutaneous insertion site.

In the antimicrobial epidermal device 100, the circumferential body 110 is adapted for epidermal placement on the treatment region 50 of a larger epidermal surface 10. Placement is based on a central void 120 in the circumferential body for epidermal access and alignment generally over the insertion site 52. The circumferential body 110 includes an illumination source disposed for emitting a therapeutic light on the treatment region 50 defined by the central void 120. An adhesive member 116, such as a patch or bandage, adheres the circumferential body 110, vessel 140 and a percutaneous penetration member such as a needle to the epidermal area around the treatment region 50.

FIG. 2 is a perspective view of the medical device of FIG. 1 for antimicrobial light treatment around a percutaneous insertion site 52. FIG. 2 shows the central void 120 accessible by a vessel gap 122 in the circumferential body 110 for passage of the medication vessel 140 to a penetration or insertion member defining the insertion site 52. The treatment region 50 is defined by a radius around the insertion site roughly centered within the circumferential body. In the example of FIG. 2, the central void 120 remains covered by an insert 121 except at the vessel gap 122 for permitting vessel access into an illumination cavity 118.

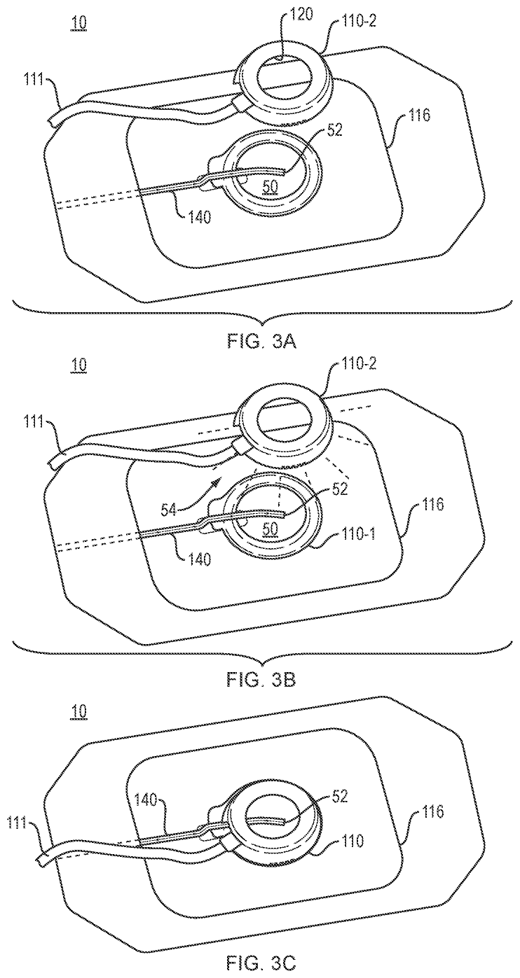

FIGS. 3A-3C show engagement of the device of FIG. 2 with a treatment region defined by the percutaneous insertion site. In a particular configuration, the device may be combined with an adhesive member 116 such as a sheet, patch or bandage for providing a system of secure attachment of the illumination source to the percutaneous insertion site. Referring to FIGS. 1-3C, the circumferential body 110 is disposed on an epidermal surface 10, in conjunction with an adhesive member 116. The adhesive member 116 has an adhesive attraction to the epidermal surface 10 and extends over the treatment region 50 and is disposed for securing the circumferential body 110 and a treatment vessel 140 directed to the central void 120. The adhesive member my include a securement or fixation dressing having adhesive and therapeutic or antimicrobial properties. The securement or fixation dressing is disposed between the circumferential body 110 and the epidermal surface 10, The circumferential body is therefore disposed in place by the underlying securement or fixation dressing/patch, and substantially centered around the insertion site.

The configuration of FIG. 3 shows a two-part configuration of the device. The circumferential body 110 further includes a distal layer 110-2 including a power connection 111 for powering an illumination source such as one or more LED elements and a proximate layer 110-1 having a translucent surface, in which the LED elements are disposed within the distal layer 110-2 for directing the therapeutic light onto the treatment region 50.

The proximate layer 110-1 engages with the adhesive member 116, which may be integrated as an adhesive whole or applied in separate phases. In the configuration of FIGS. 3A-3C, the adhesive member 116 may reside between the proximate layer 110-1 and distal layer 110-2. The adhesive member 116 secures the insertion member at the insertion site 52 alone with the medication vessel 140, shown in FIG. 3A. The treatment region 50 is defined by a radius around the dermal insertion site 52, where the insertion site 52 provides the dermal access for medical intervention through the skin by a sharp, piercing structure.

In FIG. 3B, the distal layer 110-2 approaches the secured, proximate layer 110-1. The distal layer 110-2 may already be emitting light 54 onto the treatment region 50. In FIG. 3C, the distal layer 110-2 engages the proximate portion 110-2 to form the full circumferential member 110, and encapsulates an illumination cavity, discussed further below.

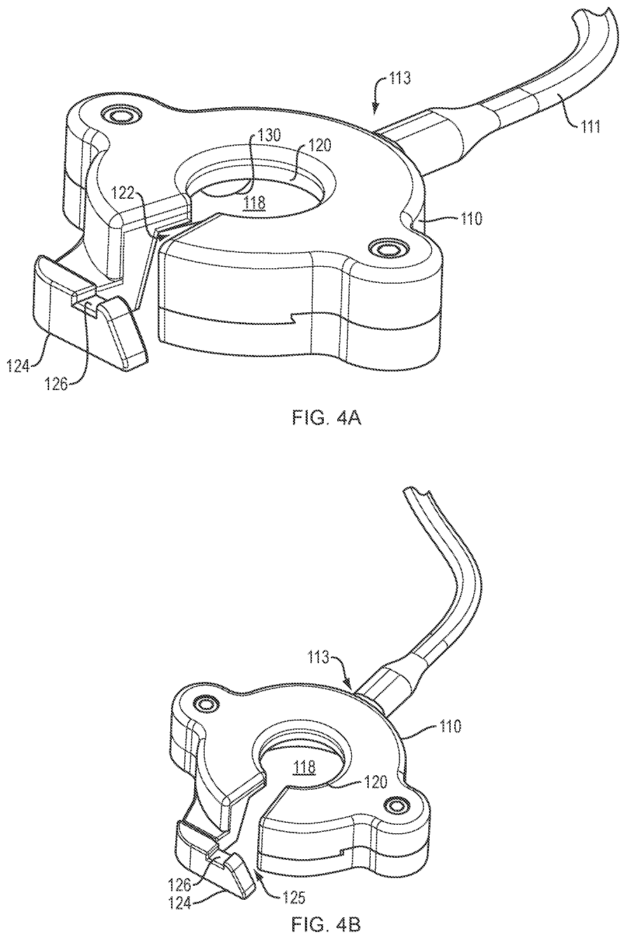

FIGS. 4A-4B show perspective views of a central void 120 in the device of FIGS. 1-3c. Referring to FIGS. 1-4B, upon adherence and proper administration, the circumferential body 110 adheres to the epidermal surface 10 with the central void 120 roughly centered on the insertion site 52. An illumination source 130 includes at least one LED element defining the illumination source, in which the LED element emits a wavelength based on an antimicrobial effect. The central void 120 has a size based on a treatment vessel 140 size and clearance over the insertion site 152. The treatment vessel 140 has an attachment to the insertion member such as a needle for a percutaneous insertion under the central void. The vessel extends through the vessel gap 122 and through the central void 120 or at least through the gap 122 and into the illumination cavity 118.

A power connection 113 receives the power supply 111 on the circumferential body 110. The power supply couples to the illumination source 130 and is adapted for receiving an electrical source for powering the illumination source, such as an external USB, batteries, AC or similar AC or DC source based on the electrical requirements of the illumination source 30. A discontinuity in the circumferential body defines the vessel gap 122 for accommodating the treatment vessel 140. The treatment vessel 140 couples to the percutaneous insertion member in the treatment region 50 under the central void 120. Routing of the treatment vessel 140 is provided by a protrusion 124 extending outward from the circumferential body. The protrusion 124 has an elevated surface 126 disposed away from the epidermal surface 10, such that the elevated surface 126 is adjacent the vessel gap 122 for directing the treatment vessel at an offset distance from the dermal surface 10. Elevation of the treatment vessel 140 above the skin avoids a shadow from the light and instead allows a shadowed region 125 to be reached by light from the illumination source 130 rather than being shaded or obscured by the vessel 140 from reaching the skin at the shadowed region.

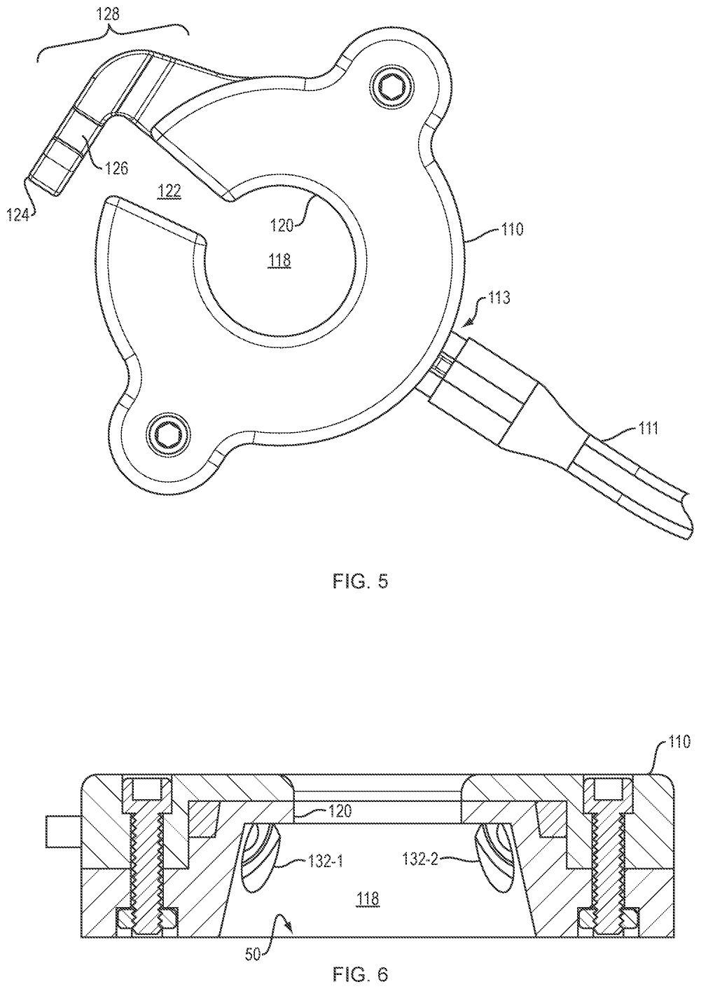

FIG. 5 shows a plan view of the device of FIGS. 1-4B. Referring to FIGS. 1-5, the vessel gap 122 is an opening or passage in the circumferential body 110. A lateral extension 128 extends radially from the circumferential body 110 adjacent the vessel gap 122, and turns toward the gap 122 to provide the elevated surface 126 residing on the protrusion 124. The elevated surface 126 is disposed on a path towards the central void 120 for receiving a treatment vessel 140 disposed on the path for fluidic delivery to an insertion site 52 in the treatment region 50.

FIG. 6 shows a side, cutaway view of the central void and light cavity formed by the device of FIGS. 1-5. Referring to FIGS. 1-6, a plurality of LED elements 132-1 . . . 132-2 (132 generally) surround the illumination cavity 118, although as few as 1 could be provided. In the example configuration, the plurality of LED elements 132 are disposed generally in a circle around the circumferential body 110, and fill the illumination cavity 118 with light focused on the treatment region 50. The inner surface of the circumferential body 110 and optional insert 121 are a light color and may be translucent to reflect and refract (distribute and target) as much if the light as possible around the illumination cavity 118 to fall on the treatment region 50. The antimicrobial light is therefore specifically targeted to fall on the treatment region defined by the percutaneous insertion and surrounding epidermal region, specifically within the illumination cavity 118 of the circumferential body 110.

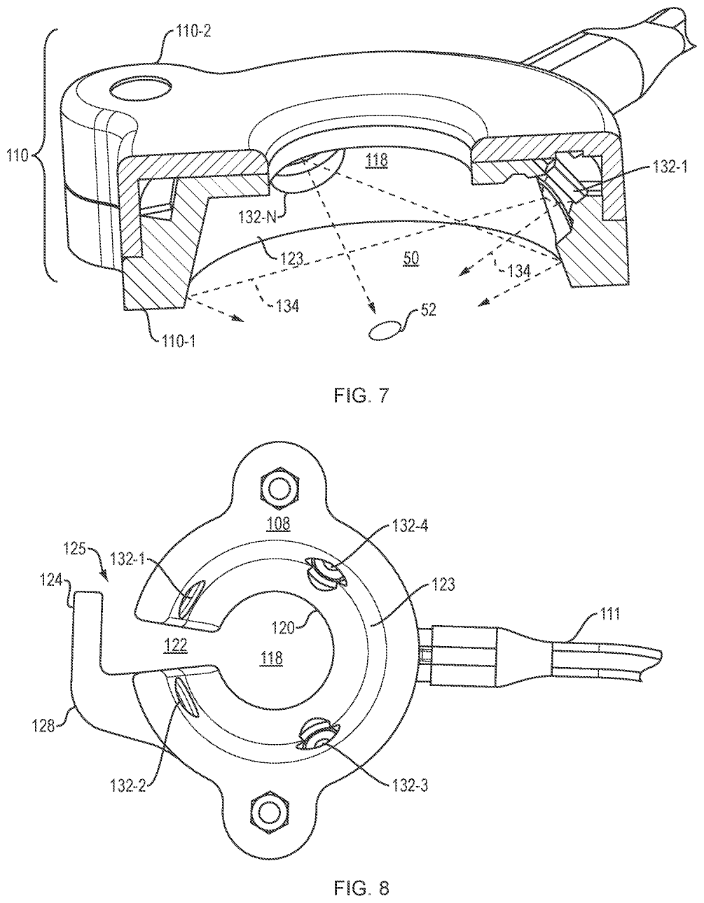

FIG. 7 shows a perspective view of the light cavity of FIG. 6 as a cutaway from the circumferential body 110. Referring to FIGS. 1-7, the circumferential body 110 is disposed on a treatment region 50 and centered on or around an insertion site 52 of a percutaneous insertion member. One or more LED elements 132-N disposed on an inner surface 123 of the circumferential body bathe the illumination cavity 118 in light for directing the light directly on the treatment region 50 and also reflected and/or refracted around the inner surface 123 as shown by arrows 134. A light colored, translucent and/or reflective property of the inner surface 123 generally focuses direct and indirect light onto the treatment region 50 for eliminating harmful pathogens that may live on the skin surface around the insertion site 52.

FIG. 8 shows a bottom view of the device of FIGS. 1-7. Referring to FIGS. 7-8, FIG. 8 shows four LEDs 132-1 . . . 132-4 emanating from the inner surface 123, however any suitable number of LEDs may be provided based on the intensity and wavelength of the therapeutic light sought for irradiation. Any suitable propagated wavelength of the electromagnetic spectrum may be provided if an illumination element can be so equipped. The underside 108 rests on the dermal surface 10 at the treatment region, adhered by the adhesive member 116 as discussed above. The protrusion 124 has a bottom flush with the underside 128, and opens to define the illumination cavity 118. The lateral extension 128 is flush with the underside 128 for resting on the skin surface, and extends in an articulated manner for protrusion 124 to form the elevated surface 126 at the vessel gap 122.

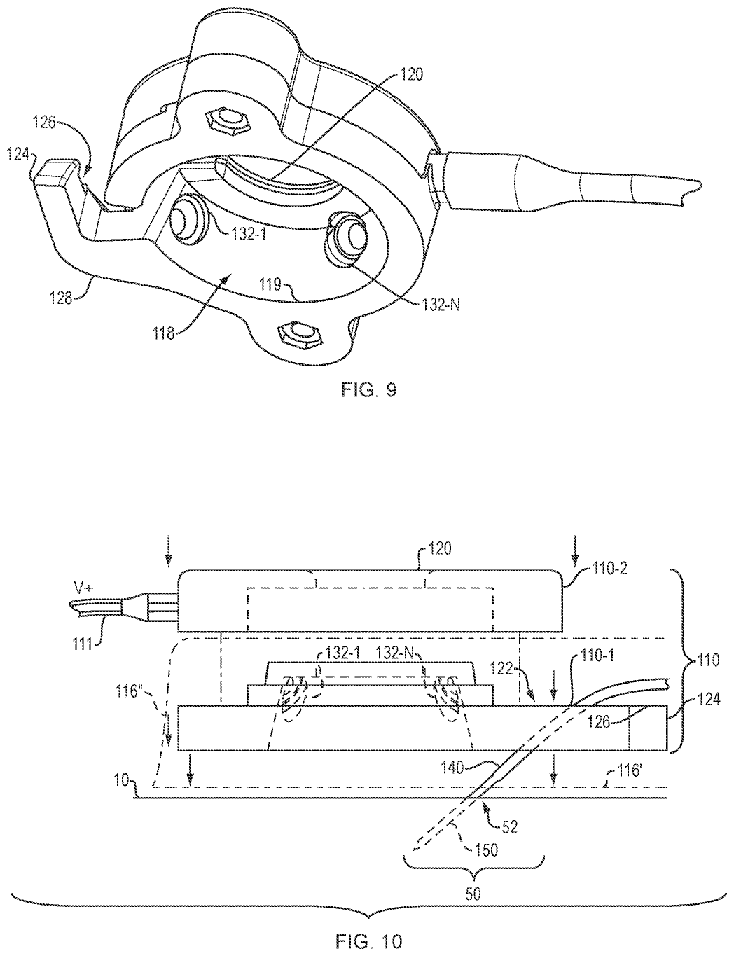

FIG. 9 shows an underside perspective view of the device and illumination cavity 118 of FIGS. 6-8. The illumination cavity 118 is based on a generally concave region under the central gap 120 and extending to an inner perimeter 119 of the circumferential body 110, with the vessel gap 122 allowing passage of the treatment vessel 140.

FIG. 10 shows a method of applying the antimicrobial light treatment of FIGS. 1-9. Referring to FIGS. 1-10, a method for antimicrobial light treatment of a percutaneous insertion site as shown in FIG. 10 includes applying an adhesive member 116 to a treatment region 50 for securing a percutaneous insertion member in an insertion site. The percutaneous insertion member 150, such as a needle, is in fluidic communication with a medication vessel 140 for delivering medication, typically an IV line, infusion line or similar delivery system. The adhesive member 116 may adhere on the epidermal surface, shown as dotted line 116′, or may be applied over the circumferential member 110, shown as dotted line 116″. In the alternate configuration of FIGS. 3A-3B, the adhesive member 116″ may reside between the proximate layer 110-1 and distal layer 110-2.

In either configuration, the circumferential body 110 is disposed onto the treatment region 52. The circumferential body 110 extends generally circular around a central void 120, and placement centers the central void around the insertion site so that the central void allows clearance for the medication vessel 140 and any uninserted portion of the rigid insertion member. The circumferential body 110 may be any suitable shape and size based on the treatment region 50 and the intensity of the illumination source 130 thereby irradiating the treatment region.

The circumferential body 110 includes a discontinuous portion defining the vessel gap 122, which may be continuous with the central void 120. In conjunction with placement of the circumferential body 110, the medication vessel is routed over the elevated surface 126 on the protrusion 124 extending from the circumferential body for permitting the vessel to extending through the vessel gap 122 above and out of contact with the skin surface. The treatment region 50 is illuminated from one or more LEDs (Light Emitting Diodes) 132 disposed on an inner surface of the circumferential body 110 for irradiating an illumination cavity 118 defined by the inner surface and the central void. The LEDs 132 or other illumination source irradiate the treatment region for maintaining an antimicrobial and sterile environment around the insertion site 50. This prevents pathogens from entering the patient along the insertion member 150.

In a particular configuration shown in FIGS. 3A-3C above, the circumferential body has multiple, engageable parts. A first, proximate layer 110-1 accompanies the insertion member 150. Disposing the circumferential body 110 further comprises disposing the proximate layer 110-1 by applying a proximate layer centered on the treatment region using the adhesive member 116, and engaging the distal layer 110-2 onto the proximate layer 116-1 by circumferentially aligning the distal layer with the proximate layer, the LEDs directed towards the illumination cavity. Any suitable adhesive, friction, interference and/or deformable (i.e. snap-fit plastic tab) mechanism may be employed for engaging the proximate 110-1 and distal 110-2 layers.

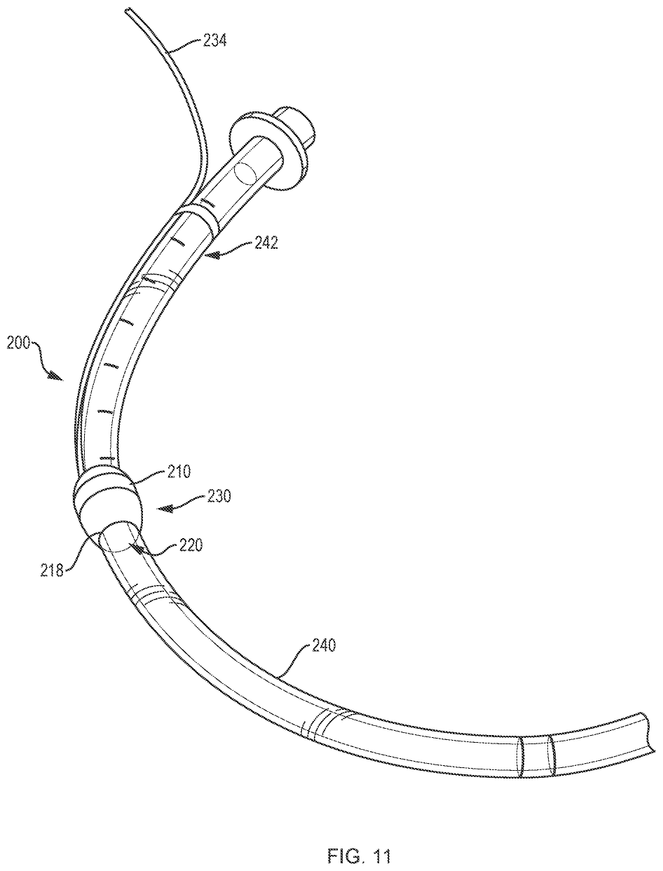

FIG. 11 shows an alternative configuration of the safety device for use with an oropharyngeal device. Irradiation of pathogenic infiltration paths may be implemented in a variety of configurations for focusing antimicrobial light in the ultraviolet or visible light spectrum. In FIG. 11, an antibacterial oropharyngeal device 200 includes a circumferential frame 210 having an array of lights 230 or other illumination source around a perimeter and directed for focusing on a central void 220 for illuminating a oropharyngeal vessel 240 passing through the central void 220.

In usage, the antibacterial oropharyngeal system therefore invokes a circumferential frame 210 having a central void 220 and at least one illumination element 232 disposed on a perimeter and directed for focusing on the central void 220. Looking further at FIG. 11, the oropharyngeal device 200 is antibacterial safety device for use with an oropharyngeal vessel 240 including the circumferential frame 210 and central void 220 adapted to receive a ventilation vessel, discussed further below. An annular exterior surface 214 on an outer circumference 218 of the circumferential frame 210 is configured for slidable, noninterfering insertion into an airway for seamless usage in conjunction with insertion of a ventilation vessel. An array 230 of illumination elements in the circumferential frame each have a transparent and flush engagement with the annular exterior surface 214 such that the illumination elements are directed tangentially along a surface of the ventilation vessel for emitting light of a predetermined antibacterial wavelength along the ventilation vessel for providing a 360° coverage against pathogens. Protection of this infiltration path is particularly effective against ventilator associated pneumonia.

The circular frame may be formed from a light conductive medium including light-transmissive materials selected from one or more of polymethyl methacrylate (PMMA), silica/quartz, thermoplastic polyurethane (TPU), flexible acrylic, transparent polyvinyl chloride (PVC), UV-inhibitor-free transparent PVC and solar cell material. The illumination elements may be disposed within the light transmissive medium or disposed on the surface of the frame.

A power supply activates the array of lights based on a connection to the power supply, and may be battery operated a tethered connection for supplying power to the LEDs. The tether 234 is aligned adjacent the annular exterior surface 242 and extends in a longitudinal direction along the ventilation vessel 240.

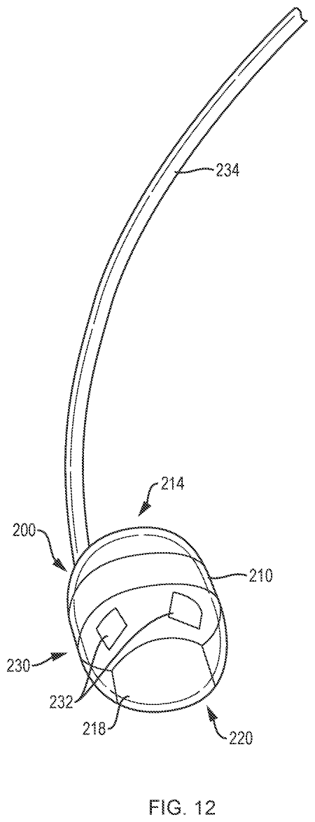

FIG. 12 is a perspective view of the oropharyngeal device of FIG. 11. The general posturing of the illumination source is for irradiation of the vessel 240 passing through the central void 220. The central void 220 has a circular or slightly elliptical cross section adapted for slidable engagement with the ventilation vessel 240, which itself as a substantially circular cross section for patient insertion. The circumferential frame 210 then forms a semicircular cross section defining the annular exterior surface 214 of the frame 210, to allow for smooth insertion without edges or ridges that could complicate or irritate insertion.

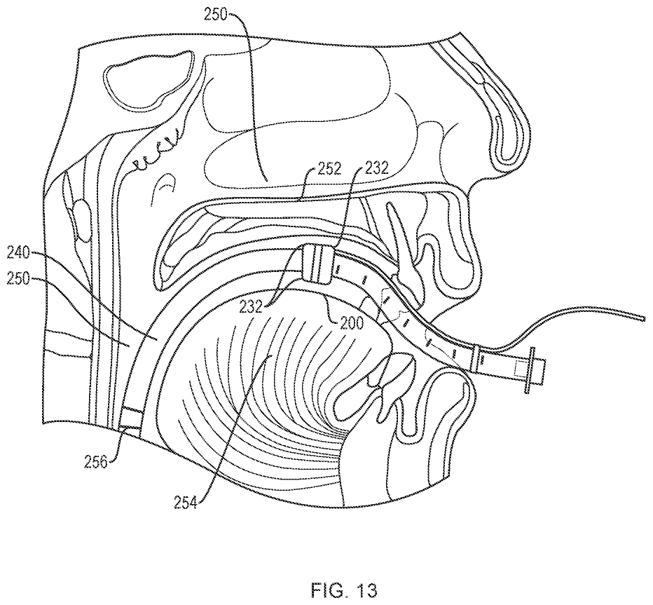

FIG. 13 is a side view of a deployed device as in FIGS. 11-12. A typical usage is in conjunction with a ventilator for respiratory support. Ventilator operation involves insertion of the oropharyngeal vessel 240 into a throat and airway region 250 for displacing and arranging soft tissue therein. Insertion is a rather invasive medical procedure undertaken by skilled doctors or medical technicians, often with a sedated patient/victim, but generally involves insertion between the palate 252 and tongue 254 for passage to a terminus at the airway 256. Such example usage may be described in greater detail by a variety of sources of competent medical authority. The oropharyngeal vessel 240 takes a subtle curvature for conforming to the anatomical structures, although the airflow and light are substantially normal to a cross section at a given point of the subtle curvature.

The illumination source typically includes one or more light emitting diodes (LEDs) 232, such that each LED provides an antimicrobial and safe light emission having a wavelength around 222 nm or 405 nm, which are wavelengths capable of eradicating pathogens and bacteria but which are not harmful to humans.

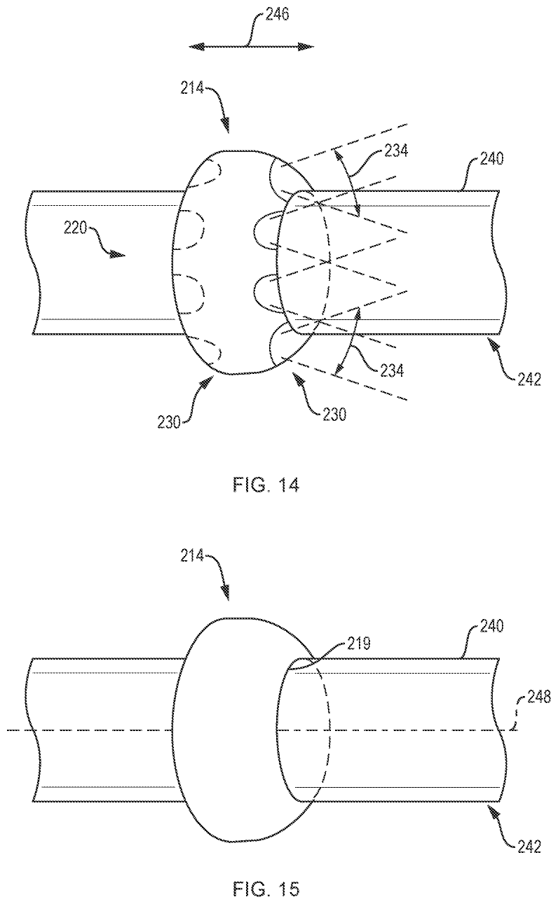

FIG. 14 is a side transparent view of the illumination sources of FIGS. 11-13. Referring to FIGS. 11-14, each of the illumination elements 232 (LEDs) is focused for irradiating the exterior surface 242 of the ventilation vessel 240. The illumination elements are focused substantially parallel to an axial direction 246 of the airflow though the vessel 240 at any given point. A dispersion range 234 resulting from a fan-out tendency of the light will cause at least some component of the light to fall on the exterior surface 242. The LEDs 232 may also be focused at a slight angle towards the surface 242 while still maintaining a flush orientation with the annular exterior surface of the frame 210. The 360° coverage of the light array 230 around the vessel 240 ensures that at least some of the range 234 will fall on the vessel surface 242 even as the vessel follows a curvature of the patient anatomy.

FIG. 15 is a side schematic view showing axial orientation of the device as in FIGS. 11-14. Referring to FIGS. 11-15, the array 230 of illumination elements 232 is directed axially 246 along the ventilation vessel 240. The array 230 of illumination elements is therefore directed substantially parallel to a longitudinal axis of the ventilation vessel 240, such that the longitudinal axis defined by a line normal to a cross section of the ventilation vessel at a given point. A subtle curvature 248 follows the vessel and the axis at any given position, however the general curvature 248 is within (less than) the dispersion range 234, or “fan out” of the dispersed illumination. An inner circumference 219 maintains contact with the exterior surface 242 for ensuring slidable engagement, while the outer circumference maintains a smooth, semicircular shape or cross-section and non-obstructing diameter so that the frame 210 can fit in the narrow insertion region 250.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. An antimicrobial light-emitting device, comprising:
an illumination source configured around an endotracheal tube for dispersing safe, antimicrobial light throughout the oropharynx.

2. The device of claim 1 wherein the light has the ability to eradicated pathogenic microorganisms without harming the patient.

3. The device of claim 1 wherein the illumination source includes one or more light emitting diodes (LEDs), each providing safe, antimicrobial light.

4. The device of claim 1 wherein the device is disposed for irradiation of the oropharynx

5. The device of claim 1 wherein the illumination source provides irradiation throughout the intubated oropharynx.

6. The device of claim 1 wherein the device consists of light transmissive material.

7. The device of claim 1 wherein there is a central void within the device adapted to engage a endotracheal tube.

8. The device of claim 7 wherein the device is slidable and can be positioned within the intubated oropharynx.

9. The device of claim 1 wherein the device is attachable onto the endotracheal tube.

10. The device of claim 1 wherein the device is integrated into the endotracheal tube.

11. The device of claim 1 wherein the light wavelength is far UVC or visible blue light.

12. An antimicrobial light emitting device comprising a light conductive medium formed from a material selected from the group of light-transmissive materials consisting of polymethyl methacrylate (PMMA), silica/quartz, thermoplastic polyurethane (TPU), flexible acrylic, transparent polyvinyl chloride (PVC), UV-inhibitor-free transparent PVC and solar cell material.

13. A method including decontaminating the intubated oropharynx comprising bathing the oropharynx in safe, antimicrobial light.

14. A method of reducing the risk of ventilator-associated pneumonia by decontaminating the oropharynx using a safe, antimicrobial light-emitting device.

15. The device of claim 1, further comprising:
a circumferential frame having a central void housing the illumination source, the central void adapted to receive a ventilation vessel;
an annular exterior surface on an outer circumference of the circumferential frame, the annular exterior surface configured for slidable, noninterfering insertion into an airway; and
the illumination source including an array of illumination elements in the circumferential frame; each of a plurality of illumination elements in the array having a transparent and flush engagement with the annular exterior surface, the illumination elements directed tangentially along a surface of the ventilation vessel and emitting light of a predetermined wavelength.

16. The device of claim 15 wherein the array of illumination elements is directed for projection axially along the ventilation vessel.

17. The device of claim 15 wherein the illumination source is disposed for irradiation of the ventilation vessel passing through the central void.

18. The device of claim 15 wherein the central void has a circular or elliptical cross section adapted for slidable engagement with the ventilation vessel, the circumferential frame having a semicircular cross section defining the annular exterior.
